(19) United States
(12) Patent Application Publication
(10) Pub. No.: US 2024/0207566 A1
(43) Pub. Date: Jun. 27, 2024

(54) SYSTEMS AND METHODS FOR PROVIDING TREATED AIR FOR A USER OF A RESPIRATORY THERAPY SYSTEM

(71) Applicant: ResMed Digital Health Inc., San Diego, CA (US)

(72) Inventors: Colleen MACNAUGHTON, Halifax (CA); Dave WILLIFORD, San Diego, CA (US); Mark Thomas FELCANSMITH, San Diego, CA (US); Michael SCANNELL, Dublin (IE); Aoibhe Jacqueline TURNER-HEANEY, Dublin (IE); Cindy Ann CHEN, San Diego, CA (US); Dylan Hermes da Fonseca BEADLE, Dublin (IE)

(21) Appl. No.: 18/391,121

(22) Filed: Dec. 20, 2023

Related U.S. Application Data
(60) Provisional application No. 63/476,527, filed on Dec. 21, 2022.

(51) Int. Cl. A61M 16/10; A61M 16/00; A61M 16/06

(52) U.S. Cl. CPC A61M 16/107; A61M 16/024; A61M 16/06; A61M 16/1045; A61M 2202/0042; A61M 2205/502; A61M 2205/8206; A61M 2230/40

(57) ABSTRACT

A wearable air treatment system includes a user interface configured to be coupled about at least a portion of a face of a user. The wearable air treatment system also includes an air treatment module coupled to the user interface. The air treatment module in turn includes an air inlet configured to receive ambient air therethrough. A light source is positioned distal of the air inlet and is configured to emit light to treat at least a portion of the received ambient air. The air treatment module also includes an air outlet positioned distal of the light source. The air outlet is configured to direct at least a portion of the treated ambient air towards an interior of the user interface.

### CROSS-REFERENCE TO RELATED APPLICATIONS

This application claims the benefit of, and priority to, U.S. Provisional Patent Application No. 63/476,527 filed on Dec. 21, 2022, which is hereby incorporated by reference herein in its entirety.

### TECHNICAL FIELD

The present disclosure relates generally to systems and methods for delivering treated air to a user, and more particularly, to systems and methods for delivering treated air to a user through an air pathway of a respirator system.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. Individuals that use these respiratory therapy systems during sleep thereby spend a significant amount of time breathing air that is provided by the respiratory therapy systems. While conventional respiratory therapy systems are able to adjust the condition (e.g., pressure, humidity, etc.) of air provided to a user for inhalation, they are unable to improve the quality of the air provided to the user. Accordingly, conventional respiratory therapy systems are often limited by the quality of the ambient air in their surrounding environment. At times, this leads to situations where users of these conventional respiratory therapy systems are forced to inspire conditioned air of an undesirable quality. The present disclosure is directed to solving these and other problems.

### SUMMARY

According to some implementations of the present disclosure, a wearable air treatment system includes a user interface configured to be coupled about at least a portion of a face of a user. The wearable air treatment system also includes an air treatment module coupled to the user interface. The air treatment module in turn includes an air inlet configured to receive ambient air therethrough. A light source is positioned distal of the air inlet and is configured to emit light to treat at least a portion of the received ambient air. The air treatment module also includes an air outlet positioned distal of the light source. The air outlet is configured to direct at least a portion of the treated ambient air towards an interior of the user interface.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3A is a perspective view of the user interface of the system of FIG. 1 showing an air treatment module removably coupled between the user interface and a conduit, according to some implementations of the present disclosure;

FIG. 3B is an exploded view of the user interface, air treatment module, and conduit of FIG. 3A, according to some implementations of the present disclosure;

FIG. 3C is a detailed perspective view of the air treatment module of FIG. 3A, according to some implementations of the present disclosure;

FIG. 3D is a perspective view of the user interface of the system of FIG. 1 showing an air treatment module removably coupled thereto, according to some implementations of the present disclosure;

FIG. 4A is a rear perspective view of the respiratory therapy device of FIG. 1 showing an air treatment module removably coupled between the respiratory therapy device and a conduit, according to some implementations of the present disclosure;

FIG. 4B is an exploded rear perspective view of the respiratory therapy device of FIG. 1 showing the alignment between the air treatment module and the conduit, according to some implementations of the present disclosure; and

FIG. 5 is a process flow diagram for a method for indicating an improvement to the quality of air inspired by a user as a result of implementing an air treatment module, according to some implementations of the present disclosure.

FIG. 6 illustrates an exemplary timeline for a sleep session, according to some implementations of the present disclosure;

FIG. 7 illustrates an exemplary hypnogram associated with the sleep session of FIG. 7, according to some implementations of the present disclosure; and

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

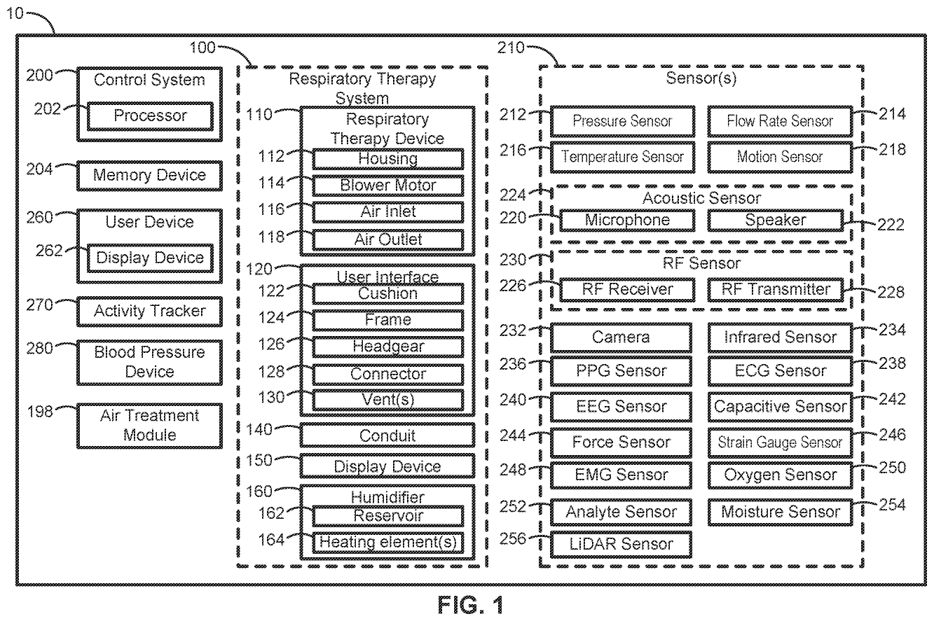

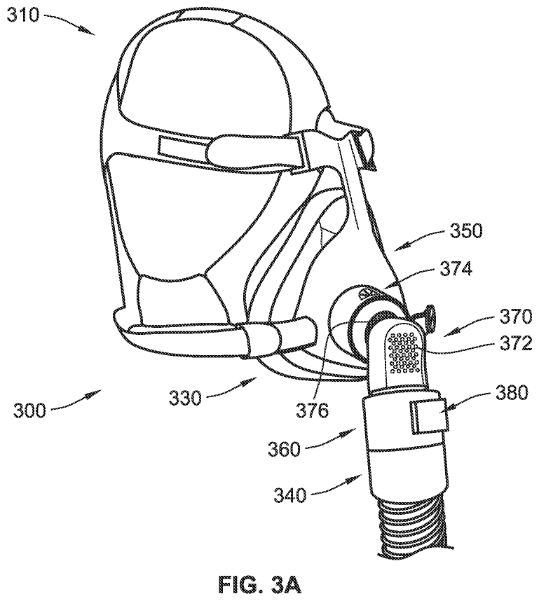

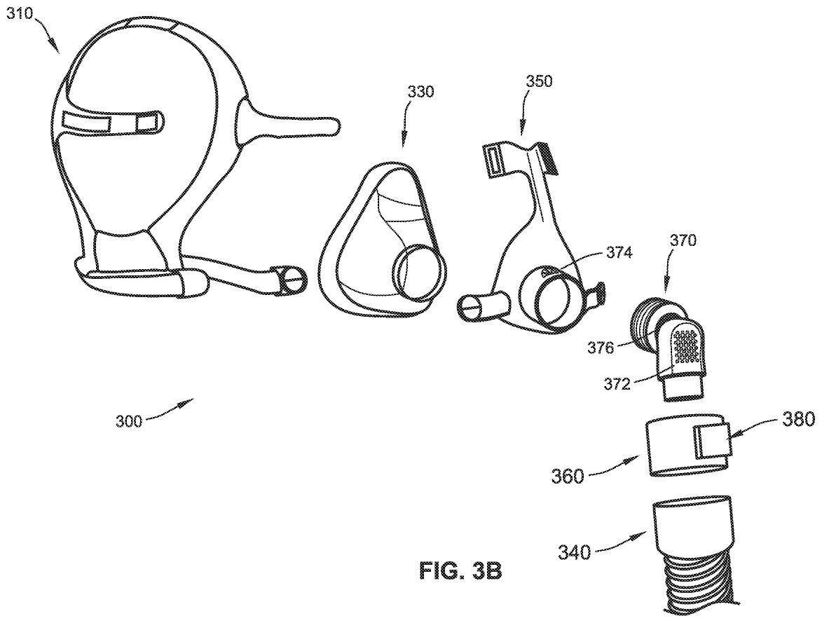

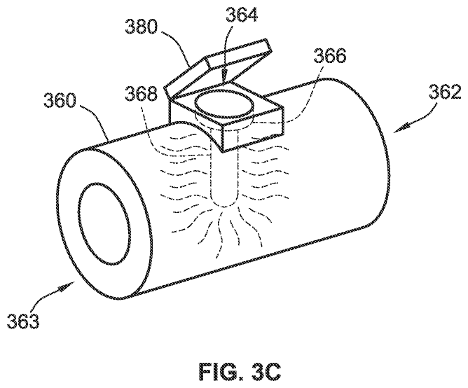

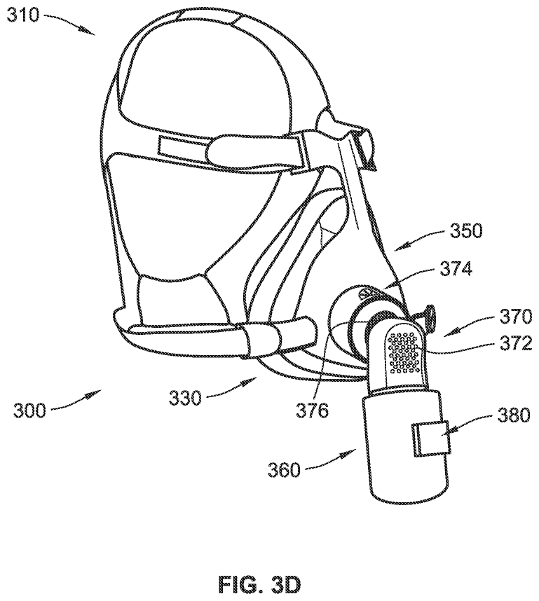

Referring to FIG. 1, a system 10, according to some implementations of the present disclosure, is illustrated. The system 10 includes a respiratory therapy system 100, a control system 200, one or more sensors 210, a user device 260, and an activity tracker 270. The system 10 further includes an air treatment module 198 which may be implemented in a number of different ways depending on the implementation. For instance, the air treatment module 198 may be coupled directly to the user interface 120 or the respiratory therapy device 110, e.g., as will be described in further detail below (e.g., see FIGS. 3A-3C).

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

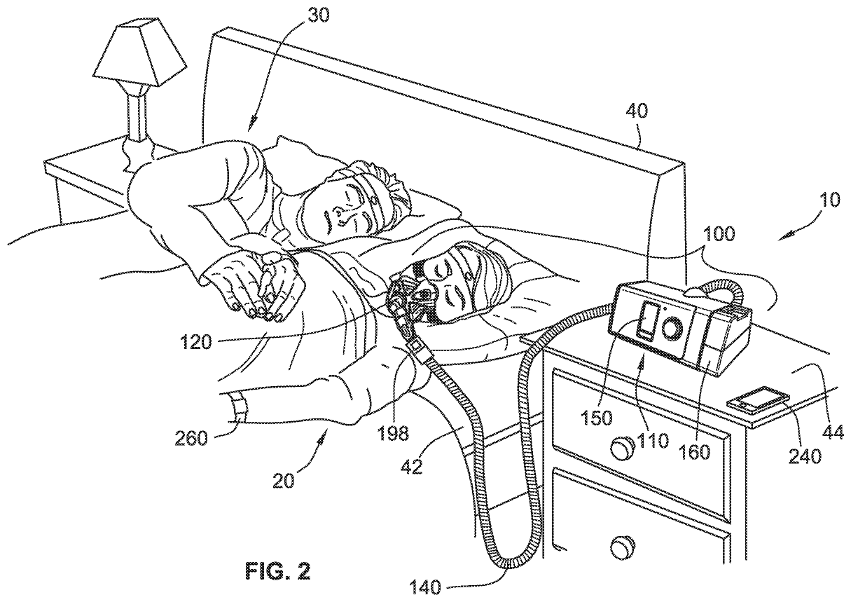

As shown in FIG. 2, the respiratory therapy system 100 can be used to treat user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

The respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure). Moreover, the air treatment module 198 is configured to treat the ambient air that is drawn into the respiratory therapy system 100, e.g., as will be described in further detail below.

Referring again to FIG. 1, the user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

Moreover, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

Referring to back to FIG. 1, the one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIG. 2), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIG. 2). Based at least in part on the data from the microphone 220 and/or the speaker 222, the control system 200 can determine a location of the user 20 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIG. 2), to determine air flow of the mouth and/or nose of the user, to determine a time when the user enters the bed (FIG. 2), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 100, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum the respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IOT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

As previously mentioned, individuals that suffer from sleep-related and/or respiratory-related disorders often treat these disorders using a respiratory therapy system (e.g., a CPAP system), which delivers pressurized air to aid in preventing the individual's airway from narrowing or collapsing during sleep. Individuals that use these respiratory therapy systems during sleep thereby spend a significant amount of time breathing air that is provided by the respiratory therapy systems.

While conventional respiratory therapy systems are able to adjust the condition (e.g., pressure, humidity, etc.) of air provided to a user for inhalation, they are unable to improve the quality of the air provided to the user. Accordingly, conventional respiratory therapy systems are often limited by the quality of the ambient air in their surrounding environment. At times, this leads to situations where users of these conventional respiratory therapy systems are forced to inspire conditioned air of an undesirable quality.

In sharp contrast to these conventional shortcomings, various ones of the implementations included herein are able to treat air in addition to conditioning the air. In other words, implementations herein are able to improve the quality of the air provided to a user while using a respiratory therapy system. It follows that users of these respiratory therapy systems are provided air for breathing that is of a desired condition (e.g., pressure, humidity, temperature, etc.) as well as a desired quality (e.g., absence of contaminants).

Some implementations even achieve these air treatment capabilities in different configurations of the respiratory therapy systems. For instance, some configurations implement the aforementioned air treatment capabilities in a respiratory therapy system having a user interface, conduit, and respiratory therapy device (e.g., see 100 of FIG. 2). In other implementations, the air treatment capabilities are achieved by a simplified configuration, e.g., as will soon become apparent.

Referring now to FIGS. 3A and 3B, a user interface 300 that is the same as, or similar to, the user interface 120 (e.g., see FIG. 1) according to some implementations of the present disclosure is illustrated. Moreover, FIGS. 3A-3C include an air treatment module 360 and conduit 340 that may be the same as, or similar to, the air treatment module 198 and conduit 140 illustrated in FIGS. 1-2. As an option, the present air treatment module 360 may be implemented in conjunction with features from any other implementation listed herein, such as those described with reference to the other FIGS. However, such air treatment module 360 and others presented herein may be used in various applications and/or in permutations which may or may not be specifically described in the illustrative implementations listed herein. Further, the air treatment module 360 presented herein may be used in any desired environment. Thus FIGS. 3A and 3B (and the other FIGS.) may be deemed to include any possible permutation.

Looking specifically now to FIGS. 3A-3B, the user interface 300 generally includes a cushion 330 and a frame 350 that define a volume of space around at least a portion of the face of the user. For instance, in some implementations the cushion 330 and a frame 350 define a volume of space around only a mouth of the user, while other implementations the cushion 330 and a frame 350 define a volume of space around only a nose of the user. In still other implementations, the cushion 330 and a frame 350 that define a volume of space around both the mouth and nose of the user, e.g., as seen in FIGS. 2 and 3A-3B.

When in use, the volume of space receives pressurized air for passage into the user's airways. In some implementations, the cushion 330 and frame 350 of the user interface 300 form a unitary component of the user interface. The user interface 300 can also include a headgear 310, which generally includes a strap assembly and optionally a connector 370. The headgear 310 is configured to be positioned generally about at least a portion of a user's head when the user wears the user interface 300. The headgear 310 can be coupled to the frame 350 and positioned on the user's head such that the user's head is positioned between the headgear 310 and the frame 350.

In some implementations, the connector 370 includes one or more vents 372 (e.g., a plurality of vents) located on the main body of the connector 370 itself and/or one or more vents 376 (“diffuser vents”) in proximity to the frame 350. These vents 372, 350 permit the escape of carbon dioxide (CO₂) and other gases exhaled by the user. In some implementations, one or more vents (similar to vents 372 and/or 376) may be located in the user interface 300. For example, some implementations include one or more vents in the frame 350 and/or in the conduit 140 itself. The frame 350 may also include at least one anti-asphyxia valve (AAV) 374, which allows CO₂ and other gases exhaled by the user to escape in the event that the vents (e.g., the vents 372 or 376) fail when the respiratory therapy device is active. In general, AAVs (e.g., the AAV 374) are present for full face user interfaces (e.g., as a safety feature). However, the diffuser vents and vents located on the mask or connector are not necessarily both present. For example, some masks might have only the diffuser vents such as the plurality of vents 376, while other masks might have only the plurality of vents 372 on the connector itself. It should also be noted that the various diffuser vents and/or vents may include an array of orifices in corresponding material itself, a mesh made of a fabric, etc.

The cushion 330 is positioned between the user's face and the frame 350 to form a seal on the user's face. While the optional connector 370 is configured to be coupled to the frame 350 and/or cushion 330 at one end and to the conduit 140 at the other end, the connector 370 is also preferably configured to be removably coupled to an air treatment module 360. In this arrangement, the air treatment module 360 is removably coupled between the conduit 140 and the user interface 300, such that the air treatment module 360 is in fluid communication with the air pathway. As the conditioned (e.g., pressurized) air flows along the conduit 140, the air flows through the air treatment module 360 and eventually into an interior of the user interface 300.

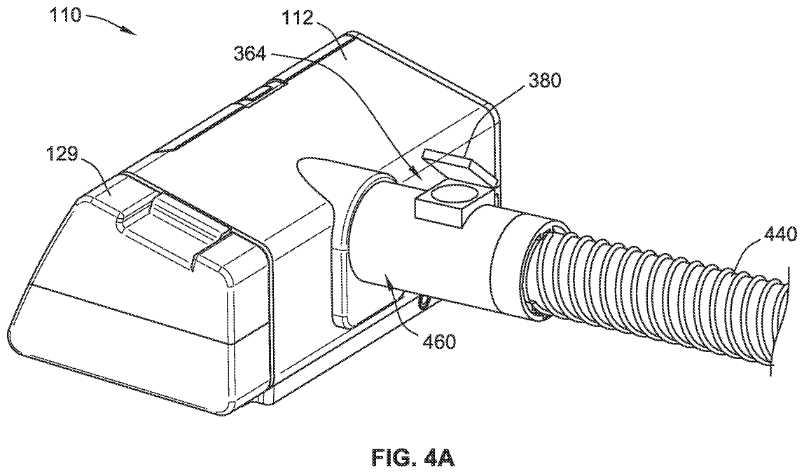

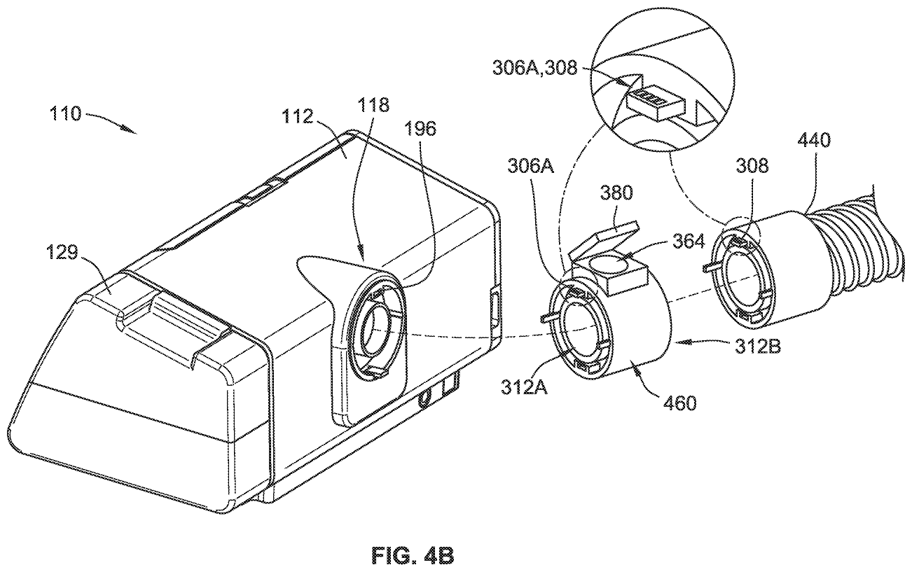

In other implementations however, the air treatment module 360 can be configured in different arrangements. For example, the air treatment module 360 may be removably coupled between the conduit 140 and a respiratory therapy device 110 (e.g., see FIGS. 4A-4B below). In other implementations, the user interface 300 may not include the connector 370. Accordingly, the air treatment module 360 and/or conduit 140 of the respiratory therapy system may be configured to connect directly to the cushion 330 and/or the frame 350. In other words, the air treatment module 360 may be used to connect the conduit 140 directly to the user interface 300. In still other implementations, the air treatment module 360 may simply be removably coupled to the air inlet of the user interface 300. In this implementation, the air treatment module 360 can be removably coupled between the air inlet of the user interface 300 and the atmosphere outside of the user interface 300 (e.g., see FIG. 3D below). It follows that the air treatment module 360 is generally placed in-line with the air pathway such that air flows through the air treatment module 360 before being directed towards an interior of the user interface 300 for inhalation by the user.

The air treatment module 360 is preferably configured to treat air that passes therethrough. The air treatment module 360 may treat the air by removing contaminants (e.g., particles, unwanted gases, pollutants, bacteria, etc.) from the air. Once treated, the air having an improved quality is directed towards the volume of space defined by the cushion 330 (or cushion 330 and frame 350) of the user interface 300 through the connector 370. From the user interface 300, the pressurized air reaches the user's airway through the user's mouth, nose, or both, e.g., as will be described in further detail below.

With continued reference to FIGS. 3A and 3B, a first end 362 of the module 360 is configured to connect to the conduit 340. The first end 362 of the module 360 and an end of the conduit 340 may connect to each other using a friction fitting, one or more releasable tabs, corresponding threaded surfaces, etc. The first end 362 of the module 360 may thereby become removably coupled to an end (e.g., air outlet) of the conduit 340, and decoupled therefrom, relatively quickly by a user. However, in some implementations the first end 362 of the module 360 may include one or more electrical contacts that are configured to mate with corresponding electrical contacts recessed in the end of the conduit 340, e.g., as would be appreciated by one skilled in the art after reading the present description. Accordingly, the air treatment module 360 may receive electrical power to operate, charge, etc., one or more electrical components that are implemented therewith.

The first end 362 of the module 360 may thereby serve as an air inlet configured to receive air therethrough. In configurations where the module 360 is removably coupled to the conduit 340, the air inlet 362 is configured to receive conditioned (e.g., pressurized, humidified, etc.) ambient air from an outlet of the conduit 140. While the air received from the conduit 140 may be conditioned, the air has not yet been treated and thereby still contains any contaminants that are present in the ambient air surrounding the respiratory therapy system. Thus, by treating conditioned air received from the respiratory therapy device, implementations included herein are desirably able to improve the quality of air that is inspired by the user. In other configurations where the module 360 is removably coupled to the user interface 300, but not the conduit 140, the air inlet 362 is configured to receive ambient air from the surrounding environment. Although the ambient air received in such configurations may not be conditioned, the module 360 is still able to treat the ambient air by removing contaminants (e.g., particles, unwanted gases, pollutants, bacteria, etc.) from the air.

Looking now to the detailed view of the air treatment module 360 in FIG. 3C, ambient air may be treated by the module 360 using a light source 368 in the present implementation. The light source 368 is shown as being positioned in an interior of the module 360, preferably such that air that passes through the module 360 comes into close proximity to the light source 368. The light source 368 is also illustrated as being positioned distal of the air inlet 362 as well as an air outlet 363, but may be positioned at any desired location in the interior of the module 360.

The light source 368 is configured to emit light therefrom that is able to treat at least a portion of the air that passes through the air treatment module 360. Accordingly, the light emitted from the light source 368 may have a specific wavelength, intensity, propagation direction, etc. that is able to remove contaminants (e.g., particles, unwanted gases, pollutants, bacteria, etc.) from the ambient air. For instance, in some implementations the light source 368 is an ultraviolet light source. Thus, the light source 368 may emit ultraviolet light having wavelengths between about 200 nanometers and about 300 nanometers, but the wavelengths could be shorter or longer depending on the desired implementation. Moreover, according to some approaches, the ultraviolet light may be emitted as part of ultraviolet germicidal irradiation (UVGI). The light emitted from the light source 368 is thereby able to treat (e.g., remove contaminants from) at least a portion of the ambient air received at the inlet 362 of the module 360. As a result, the air that is ultimately directed towards an interior of the user interface 300 for inhalation by the user is of a desired quality in addition to being of a desired condition.

A second end 363 of the air treatment module 360 is also illustrated as being removably coupled to a distal end of the connector 370. The second end 363 is also positioned distal of the light source 368. The second end 363 may serve as the outlet of the module 360. The second end 363 (e.g., outlet) is thereby configured to direct at least a portion (more preferably at least a majority) of the air that enters the module 360, towards an interior of the user interface 300. The module 360 may be removably coupled to the connector 370 using releasable clips, friction fittings, magnets, etc., or any other type of connection that would be apparent to one skilled in the art after reading the present description.

The light source 368 may be powered differently depending on the implementation. For instance, the light source 368 may be powered using one or more batteries in some implementations, e.g., as will be described in further detail below. In other implementations, a wired power supply (e.g., extending along a conduit) may be used to provide operating power for the air treatment module and/or components therein. However, it should be noted that any desired type of power supply may be used to operate the light source 368.

According to the present implementation, the air treatment module 360 defines an internal receptacle 364 that is configured to receive and contain one or more batteries 366. The internal receptacle 364 may be configured to receive any desired size of battery, e.g., such as AA, AAA, C, D, button cell, etc., depending on the implementation. Different types of batteries may also be used. For instance, in some approaches one or more rechargeable batteries may be used in the air treatment module 360. In other approaches, the air treatment module 360 may use non-rechargeable batteries.

The air treatment module 360 additionally includes a moveable cover 380 that controls access to the receptacle 364. FIG. 3A shows the cover 380 in a first position. In the first position, the cover 380 is open, such that the batteries 366 are accessible in the receptacle 364. Moreover, FIG. 3B shows the cover 380 in a second position. In the second position, the cover 380 is closed, such that the batteries 366 cannot be accessed. The cover 380 is generally illustrated as being configured to pivot between the first position and the second position, for example via one or more hinges or one or more pivots. However, the cover 380 can be configured to be moveable using any number of mechanisms or techniques. For example, the cover 380 may move in a single plane either horizontally or vertically between the first position and the second position. The cover 380 may also be configured to be twisted, such that rotation of the cover 380 moves the cover 380 between the first position and the second position. In still other implementations, the cover 380 may be attached to the air treatment module 360 via a friction fitting, a press fitting, a snap fitting, etc. In these implementations, opening the cover 380 to allow access to the batteries 366 in the receptacle 364 can include detaching the cover 380 from the air treatment module 360. Closing the cover 380 to prevent access to the receptacle 364 can include attaching the cover 380 to the air treatment module 360. The cover 380 can be manually moveable by a user, controlled by a control system (e.g., see 110 of FIG. 1), etc.

Once inserted properly in the internal receptacle 364, the one or more batteries 366 may supply electrical power to other portions of the air treatment module 360. The one or more batteries 366 may thereby provide the electrical power used to actually treat the ambient air received. For instance, the batteries 366 may provide a sufficient amount of electrical power to operate the light source 368. However, the batteries 366 may additionally or alternatively provide operating power to an ionizer purification component, a polarized-media electronic component, an immobilized cell with a bio-reactive mass, etc., which may be included in the treatment module 360 in some implementations.

It should also be noted that the air treatment module 360 may be powered by a different electrical source in other implementations. For instance, the air treatment module 360 may be coupled to a wired power supply (e.g., extending along the conduit) in some implementations. Accordingly, the air treatment module 360 may include one or more sets of electrical contacts that are capable of electrically coupling the air treatment module 360 to a portion of the respiratory therapy system. In some implementations, the electrical contacts may be able to electrically couple the air treatment module 360 to the user interface 300. The electrical contacts may also be able to electrically couple the air treatment module 360 to the conduit 340, e.g., as mentioned above.

While the air treatment module 360 includes a light source 368 configured to treat ambient air received by the air treatment module 360 in some implementations, different air treatment components may additionally or alternatively be used to treat ambient air received by the module 360. For instance, the air treatment module 360 itself may include a light source 368 in addition to another air treatment component. In other implementations, the air treatment module 360 may not include the light source 368, but rather a different type of air treatment component. In still other implementations, the air treatment module 360 may include the light source 368, while additional air treatment components are implemented elsewhere in the respiratory therapy system. Moreover, the additional air treatment component(s) may be passive air treatment component(s) and/or active air treatment component(s), e.g., as will soon become apparent.

According to an example, the air treatment module 360 itself may include a light source in addition to a physical air filter. The physical air filter may be considered a passive air treatment component and is preferably configured to remove particulates from the ambient air as it passes through the physical air filter. Depending on the type of filter used, different types of particulates may be removed from the ambient air by the physical air filter. For instance, in some implementations the physical air filter is a high-efficiency particulate absorbing (HEPA) filter that is capable of removing a higher percentage of particulates than a standard air filter. In other implementations, a user may be highly sensitive to the presence of particulates in the air they inspire and therefore an Ultra low particulate air (ULPA) filter may be utilized by the air treatment module 360. In still other implementations, the physical air filter may be an activated carbon filter positioned proximal to the air inlet of the air treatment module 360 and configured to treat at least a portion of the received ambient air. However, any desired type of physical air filter may be used.

In some implementations, the physical air filter may be integrated with the air treatment module 360. For instance, the physical air filter may be positioned proximal to the air inlet of the air treatment module 360. In other implementations, the physical air filter may be positioned proximal to the outlet of the air treatment module 360 such that the filter acts to capture any particulates that were not removed by the light source. This may also be desirable as the physical air filter will capture fewer particulates than being positioned at an inlet of the air treatment module 360, thereby leading to fewer replacements.

It follows that the physical air filter may be removably coupled to the air treatment module 360 such that the filter may be removed and cleaned, replaced, swapped out, etc., as desired over time. This also allows a user the ability to selectively install a type of air filter that is best suited for the environment and/or activity the user is in or plans to experience. The physical filter may be removably coupled to the air treatment module 360 using friction fittings, releasable clips, fasteners, etc. It should also be noted that although it may be desirable for the physical air filter to be removably coupled to the air treatment module 360, the air filter may be positioned elsewhere in the respiratory therapy system. For instance, in some implementations the air filter may be placed at an inlet of the respiratory therapy device (e.g., see 116 of FIG. 1) such that air is treated as it enters the respiratory therapy system. This may desirably reduce the impact experienced by the air treatment module 360, leading to a longer effective lifetime for the components included therein. The respiratory therapy system may even implement multiple physical air filters throughout the air pathway, e.g., depending on the desired implementation.

While some implementations include passive air treatment components, e.g., like physical air filters as mentioned above, additional active air treatment components may also be included in the respiratory therapy system in some instance. For example, the air treatment module 360 may include components that actively perform processes that remove contaminates from the ambient air taken in from the system's surroundings. These active air treatment components may be implemented in addition to the light source 368 of the air treatment module 360 in some implementations, while in others, the light source 368 may be replaced by a different type of active air treatment component. It follows that the size, shape, features, etc., of the air treatment module 360 may vary depending on the specific implementation.

For instance, in some implementations the air treatment module 360 includes an ionizer purification component. According to a specific example, the ionizer purification component is a plasma air purification component. The ionizer purification component is preferably configured to produce positive and negative ions to treat at least a portion of the ambient air received by the air treatment module 360. These bipolar ions are dispersed into the air treatment module 360 such that they proactively attack airborne contaminants in the ambient air. As would be appreciated by one skilled in the art after reading the present description, this process occurs because the bipolar ions seek out atoms and molecules in the air to exchange electrons with in an effort to restabilize, effectively neutralizing particulate matter, bacteria, virus cells, odorous gases and aerosols, etc. It follows that the ionizer purification component is positioned distal of the air inlet in some implementations. The ionizer purification component may also be powered by the battery included in the air treatment module 360.

In other implementations, the air treatment module 360 includes a polarized-media electronic component. The polarized-media electronic component is preferably configured to generate a polarized electric field to treat at least a portion of the ambient air received at the air treatment module 360. Accordingly, the polarized-media electronic component may be positioned distal of the air inlet. The polarized-media electronic component may alternatively be positioned proximal to the air inlet, proximal to the air outlet, distal of the air outlet, etc., e.g., depending on the desired configuration. In still other implementations, the air treatment module 360 includes an immobilized cell with a bio-reactive mass. The immobilized cell is preferably configured to treat at least a portion of the received ambient air. Accordingly, the immobilized cell may be positioned distal of the air inlet as well in such implementations. The immobilized cell may alternatively be positioned proximal to the air inlet, proximal to the air outlet, distal of the air outlet, etc., e.g., depending on the desired configuration.

While the air treatment module 360 is configured to be operably coupled to the conduit 140 as well as the frame 350 and/or cushion 330, the air treatment module 360 may be configured to also operate while decoupled from the conduit 140. In other words, the user interface 300 may be donned by a user and provide treated air to the user while remaining disconnected from the conduit 140. This desirably allows for a user to wear the user interface 300 and receive treated air to breath despite being disconnected from the remainder of a respiratory therapy system. Users are thereby able to continue wearing the user interface 300 and inspire treated air received from the air treatment module 360 at any time. For instance, the user interface 300 and air treatment module 360 may be decoupled from the conduit according to one implementation.

In such implementations, the air treatment module 360 may draw in ambient air directly from the surrounding environment through air inlet 362. The air treatment module 360 may also rely on batteries 366 to power any electrical components in the user interface 300 and/or air treatment module 360. For instance, the light source 368 may be powered by the batteries 366 while the air treatment module 360 is disconnected from the conduit. However, implementations having rechargeable batteries 366 may begin recharging the batteries 366 in response to the air treatment module 360 being recoupled to the conduit. For instance, a wired power supply extending along a conduit may provide the electrical power used to recharge the batteries 366 in response to the conduit being recoupled to the air treatment module 360. It should also be noted that the air treatment module 360 may be coupled directly to the cushion 330 and/or frame 350 in some implementations. Accordingly, the connector 370 may be removed from the user interface, thereby allowing the air treatment module 360 to be removably coupled directly to the cushion 330 and/or frame 350.

It follows that the implementations described herein with respect to FIGS. 3A-3C are desirably able to provide treated air to a user for inhalation regardless of whether the user is actually coupled to a respiratory therapy system. This desirably improves the quality of air inspired by users, leading to additional improvements in a user's lung health, respiratory system, and even quality of sleep, e.g., as will be described in further detail below.

Referring now to FIG. 3D, a user interface 390 that is the same as, or similar to, user interface 120 of FIGS. 1 and/or 300 of FIGS. 3A-3B, according to some implementations of the present disclosure is illustrated. Moreover, FIG. 3D includes an air treatment module 360 that is coupled to the user interface 390 at one end, while the other end of the air treatment module 360 remains exposed to the ambient environment (e.g., uncoupled). Accordingly, air treatment module 360 may be the same as, or similar to, the air treatment module 198 illustrated in FIGS. 1-3B. As an option, the present user interface 390 and air treatment module 360 may be implemented in conjunction with features from any other implementation listed herein, such as those described with reference to the other FIGS. However, such components and others presented herein may be used in various applications and/or in permutations which may or may not be specifically described in the illustrative implementations listed herein. Further, the user interface 390 and air treatment module 360 presented herein may be used in any desired environment. Thus FIG. 3D (and the other FIGS.) may be deemed to include any possible permutation.

As noted above, the air treatment module 360 is removably coupled to the user interface 390, e.g., using any of the implementations included herein. However, an opposite end of the air treatment module 360 remains uncoupled and thereby serves as an air inlet. In other words, the first end 362 of the module 360 may be configured to receive ambient air from the surrounding environment therethrough. Although the ambient air received in such configurations may not be conditioned, the module 360 is still able to treat the ambient air by removing contaminants (e.g., particles, unwanted gases, pollutants, bacteria, etc.) from the air.

In some instances, this will allow for the user interface 390 to draw in ambient air from a natural source. For example, a user may be located outdoors. This allows for the scents associated with things around the user to be enjoyed by the user. However, in other instances, it may be desirable to tailor the scent that is delivered to a user donning a user interface 390. For example, one or more artificial sources may be used to introduce a unique smell to the air being inspired by the user. These artificial sources may introduce (e.g., exude) scents that are configured to mimic the general smell of the beach, a campfire, a rainforest, calming oils, etc. Accordingly, the user interface 390 receives air that has been treated (e.g., purified) by the air treatment module 360 as well as scented to match a desired (e.g., predetermined) smell. This may enhance the quality of sleep the user experiences by providing a naturally pleasant, treated air for inspiration. Scents that are particularly pleasing for a given user may be predetermined by sending a number of scent samples to the user to choose one or more from for implementation in the respiratory therapy system, e.g., as described herein.

The desired smell may be provided during sleep onset, and could be adjusted as sleep progresses. For instance, the specific scent, strength of the scent, delivery path of the scent, etc. may change over time and/or based on feedback received. In some approaches, a scent may even be removed in certain situations. This may allow for the system to avoid situations where the user becomes nose blind and is no longer able to smell (e.g., benefit) from the presence of the scent. According to an example, two filtered air intakes may be used to receive ambient air depending on the stage of a user's sleep cycle. A first of the filtered air intakes is scented and used to provided scented, treated air to the user during the beginning of a sleep cycle. However, in response to determining that the user has actually fallen asleep, the system may switch to a second of the filtered air intakes which is not scented. The user may be determined as having fallen asleep based on respiratory data (e.g., breathing rate), movements of the user, medica data (e.g., a heart rate), time of day, etc.

The scent(s) may even be exuded in combination with one or more sounds being played over a speaker. For instance, in implementations where the user interface and air treatment module are coupled to a respiratory therapy device, a speaker of the device may play audio signals that correspond to the scents being exuded for the user to smell. According to an example, the smell of the ocean may be exuded into treated (e.g., purified) air provided to the user for inhalation while the speaker of the respiratory therapy device pays sounds of waves crashing on the shore of the beach. This gives the user a more authentic experience, and may also allow for operating sounds of the respiratory therapy system (e.g., motors) to be masked.

In other implementations, specific gasses and/or molecules may intentionally be added to the air provided to the user for inhalation. For example, the air treatment module may include a supply of oxygen that is selectively added to the ambient air after it is received at the air treatment module 360 and treated. Similarly, in some implementations the amount of carbon dioxide in the air provided to the user for inhalation may be reduced.

In other implementations, the user interface 390 and/or air treatment module 360 may include a scented air filter. The scented air filter may passively remove contaminants (e.g., particles, unwanted gases, pollutants, bacteria, etc.) from the air, while also exuding a predetermined scent into the ambient air that passes through the scented air filter. The scented air filters may even be exchanged depending on the situation.

It follows that scented air filters may be removably coupled to the user interface 390 and/or air treatment module 360 such that the filter may be removed and cleaned, replaced, swapped out, etc., as desired over time. This also allows a user the ability to selectively install a type of scented air filter that is best suited for the environment and/or activity the user is in or plans to experience. The scented air filter may be removably coupled to the air treatment module 360 using friction fittings, releasable clips, fasteners, etc. It should also be noted that the scented air filter may be implemented according to any of the implementations herein pertaining to a physical air filter.

In other implementations, a removable humidifier module may be coupled to the air treatment module. The humidifier module is able to adjust a humidity of air as it enters the air treatment module, as it is being treated by the air treatment module, as it exits the air treatment module, etc. In still other approaches, an air treatment module may be configured to deliver controlled amounts of a medication (e.g., prescribed medication, over the counter medication, natural remedies, etc.). The amount of medication may be controlled based on details of the prescription, medical data received from one or more sensors configured to capture medical data from the user (e.g., heart rate, breath rate, depth of breath, body temperature, etc.), etc. Accordingly, a determination may be made based on information (e.g., data) corresponding to the user. This information may be evaluated in real-time as it dynamically changes and adjust the amount of medication administered to the user accordingly. It follows that the air treatment module may be configured differently depending on the desired implementation.

While various ones of the implementations included herein have been described in the context of having an air treatment module coupled directly to a user interface (e.g., see FIGS. 3A-3C), the air treatment module may be implemented differently in a respiratory therapy system. For instance, one or more components may be positioned between the air treatment module and the user interface such that the air treatment module is indirectly coupled to the user interface. According to an example, a physical air filter may be removably coupled between the air outlet of the air treatment module and the air inlet of a connector of the user interface. In another example, the physical air filter may be removably coupled between the air outlet of the air treatment module and the cushion and/or frame of the user interface.

In other implementations, an air treatment module may be positioned elsewhere along the air pathway. For instance, the air treatment module may be positioned between the conduit and the respiratory therapy device. Referring now to FIGS. 4A-4B, a perspective view of the back side of the respiratory therapy device 110 is shown. Specifically, the respiratory therapy device 110 is shown as having an air treatment module 460 removably coupled directly to the air outlet 190. The conduit 440 is also removably coupled to the air treatment module 460. Accordingly, the air treatment module 460 is positioned between the conduit 440 and the respiratory therapy device 110 in the present implementation.

The air treatment module 460 may be removably coupled to the conduit 440 and/or the respiratory therapy device 110 using any one or more of the configurations described herein. Accordingly, the air treatment module 460 may be removably coupled to the conduit 440 and/or the respiratory therapy device 110 using press fit connections, snap fit connections, a threaded connection, etc. The conduit 440 may even include one or more heating elements configured to heat the pressurized air flowing through the conduit 440. The conduit 440 is thereby able to heat the air to a predetermined temperature or within a range of predetermined temperatures. Such heating elements can be coupled to and/or imbedded in the conduit 440. In such implementations, ends of the conduit 440, air treatment module 460, and/or respiratory therapy device 110 may include electrical contacts that may become electrically coupled to each other. Once electrically coupled to each other, the conduit 440 and/or air treatment module 460 may receive electrical power sufficient to operate electrical components therein. For instance, an air treatment component in the air treatment module 460 (e.g., see 368 of FIG. 3C), batteries of the air treatment module 460, the one or more heating elements of the conduit 440, etc., may receive electrical power.

Looking now specifically to FIG. 4B, the air treatment module 460 and conduit 440 are illustrated as including a set of electrically conductive contacts in accordance with one implementation. As an option, the present electrically conductive contacts may be implemented in conjunction with features from any other implementation listed herein, such as those described with reference to the other FIGS. However, such electrically conductive contacts and others presented herein may be used in various applications and/or in permutations which may or may not be specifically described in the illustrative implementations listed herein. Further, the electrically conductive contacts presented herein may be used in any desired environment. Thus FIG. 4B (and the other FIGS.) may be deemed to include any possible permutation.

The first set of electrical contacts 306A generally protrudes from the air treatment module 460. These electrical contacts 306A may be configured to mate with corresponding electrical contacts 196 recessed in the respiratory therapy device 110. The opposing side of the air treatment module 460 may further be configured to be removably electrically coupled to the conduit 440. For instance, the air treatment module 460 may include a second set of electrical contacts 306B recessed in the air treatment module 460. This second set of electrical contacts 306B of the air treatment module 460 may further be configured to be removably electrically coupled with a corresponding set of electrical contacts 308 on the conduit 440.

The electrical contacts 196, 306A, 306B, 308 may be used in a number of different ways. For example, in some implementations one or more of the electrical contacts 196, 306A, 306B, 308 may be used to charge a battery in the internal receptacle 364 of the air treatment module 460. It follows that some of the electrical contacts 196, 306A, 306B, 308 may be electrically coupled to an electrical power source, e.g., such as an internal system battery, an electrical outlet (via a power cord), an electrical generator, etc. The electrical power may be managed by a control system (e.g., see 110 of FIG. 1) such that the batteries remain sufficiently charged. For instance, the control system may detect whether the electrical contacts 196, 306A, 306B, 308 are coupled to each other, and selectively provide the electrical power in situations that the air treatment module 460 is electrically coupled to a remainder of the respiratory therapy system. Accordingly, the battery may be charged in response to the electrical contacts 196, 306A, 306B, 308 being coupled to each other, as well as a respiratory therapy device of the system being powered on. This may desirably avoid inefficiencies caused by unintentional current leak.

In other implementations, one or more of the electrical contacts 196, 306A, 306B, 308 are used to heat air flowing through the air treatment module 460 and the conduit 440. The air may be heated either directly or by heating a separate heating element that increases the temperature of the air. One or more of the electrical contacts 196, 306A, 306B, 308 can also act as a heater that is configured to heat the substance and cause some or all of the substance to evaporate, again either directly or by heating a separate heating element. The evaporation of the substance can be selectively controlled manually by a user, and/or by a control system (e.g., see 110 of FIG. 1).

It follows that one or more of the electrical contacts 196, 306A, 306B, 308 may be used to electrically couple the air treatment module 460 and/or the conduit 440 to a control system. In such implementations, the control system may be able to control the air treatment module 460. As a result, the control system may monitor performance of the air treatment module 460 and control treatment of ambient air passing therethrough. It follows that the control system may be able to control the light source 368 and/or any other air treatment components that may be included in the air treatment module 460. The control system may control operation of the air treatment module 460 based on a variety of different factors, including physiological data related to the user and/or a sleep session. The control system can also be used to control the movement of the cover 380 between the first (open) position and the second (closed) position.

The respiratory therapy device 110 is also depicted as including a housing 112 and an air outlet 118. In some implementations the respiratory therapy device 110 may further include an air inlet (not shown). While not shown in the present perspective view of the respiratory therapy device 110, the air inlet includes an inlet cover in some implementations that is moveable between a closed position and an open position. The air inlet cover may further include one or more air inlet apertures defined therein. Moreover, a blower motor of the respiratory therapy device 110 (e.g., see 114 of FIG. 1) may be configured to draw air in through the one or more air inlet apertures defined in the air inlet cover. The blower motor is further configured to cause this air to flow through the humidification tank 129 and out of the air outlet 118. It should also be noted that any desired internal tubing, ducts, channels, etc., may be used to further direct ambient air received by the respiratory therapy device 110 and/or air treatment module 460.

As previously mentioned, various implementations herein are able to provide treated air to a user for inhalation regardless of whether the user is actually coupled to a respiratory therapy device. A user is thereby able to breath treated air as desired, thereby improving the user's lung health, respiratory system, and even quality of sleep. These improvements may further be identified and presented to the user to affirm the benefits of utilizing the implementations described herein.

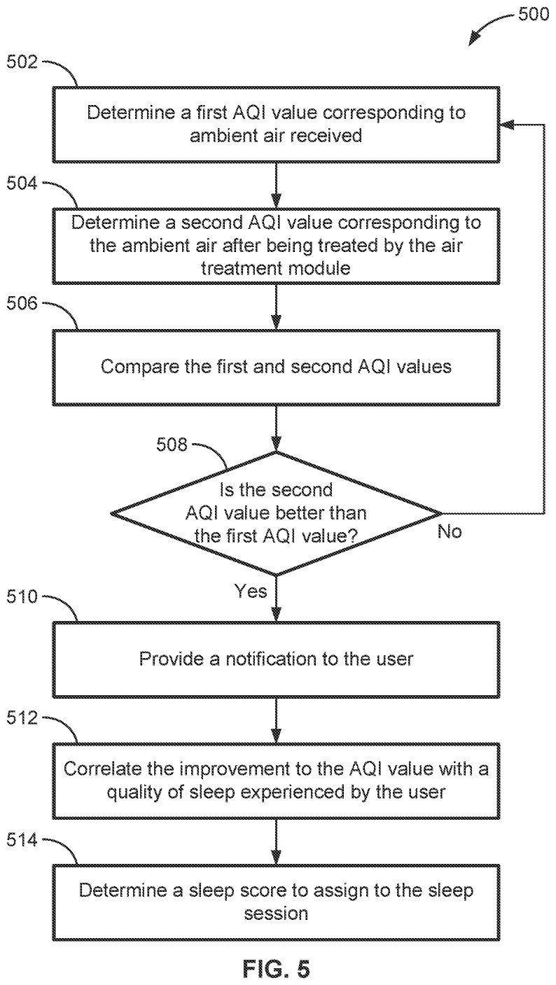

For instance, FIG. 5 illustrates a method 500 for indicating an improvement to the quality of air inspired by a user as a result of implementing an air treatment module, in accordance with one implementation. The method 500 may be performed in accordance with the present invention in any of the environments depicted in FIGS. 1-4B, among others, in various implementations. Of course, more or less operations than those specifically described in FIG. 5 may be included in method 500, as would be understood by one of skill in the art upon reading the present descriptions.

Each of the steps of the method 500 may be performed by any suitable component of the operating environment using known techniques and/or techniques that would become readily apparent to one skilled in the art upon reading the present disclosure. For example, in various implementations, the method 500 may be partially or entirely performed by a controller, a processor, etc., or some other device having one or more processors therein. The processor, e.g., processing circuit(s), chip(s), and/or module(s) implemented in hardware and/or software, and preferably having at least one hardware component may be utilized in any device to perform one or more steps of the method 500. Illustrative processors include, but are not limited to, a central processing unit (CPU), an application specific integrated circuit (ASIC), a field programmable gate array (FPGA), etc., combinations thereof, or any other suitable computing device known in the art. It follows that in some implementations, one or more of the processes described herein may be performed by a processor positioned in or in communication with an air treatment module implemented in a respiratory therapy system. Batteries included in the air treatment module may thereby be used to power a processor or other electrical components therein (e.g., such as an antenna) in some implementations.

As shown in FIG. 5, operation 502 includes determining a first air quality index (AQI) value corresponding to ambient air received at an air treatment module. In other words, operation 502 includes testing the ambient air to determine a metric that represents the quality of the ambient air. It follows that the air treatment module and/or the remainder of a respiratory therapy system in which the module is positioned may include one or more sensors capable of detecting the number of contaminants in the ambient air. For example, one or more air quality sensors may be positioned at an air inlet of the air treatment module and/or the greater respiratory therapy system. However, the AQI values may be determined using any processes that would be apparent to one skilled in the art after reading the present description. For instance, air quality data may be obtained from external sources (e.g., meteorological services that provide pollen data).

Operation 504 further includes determining a second AQI value corresponding to the treated ambient air. In other words, operation 504 includes determining the quality of the ambient air after it is treated by the air treatment module. As noted above, the air treatment module preferably removes contaminants (e.g., particles, unwanted gases, pollutants, bacteria, etc.) from the air such that the user is provided cleaner air to breath while donning a user interface coupled to the air treatment module. It follows that the second AQI value may be determined using data sampled by one or more sensors positioned proximal to an outlet of the air treatment module. In other implementations, the second AQI value may be determined using data sampled by one or more sensors positioned in the user interface.

The first and second AQI values are also compared in operation 506. By comparing the two AQI values, operation 506 can determine whether the second AQI value has improved in comparison to the first AQI value. In other words, operation 506 may include determining whether the air treatment module was able to improve the quality of the ambient air originally received. In some implementations, the determination may be made by deciding whether the second AQI value is less than the first AQI value.

Decision 508 thereby includes determining whether the second AQI value has improved compared to the first AQI value. In response to determining that the second AQI value has not improved compared to the first AQI value, the flowchart returns to operation 502 such that additional ambient air may be received and treated. It follows that processes 502, 504, 506, 508 may be repeated any number of times. However, in response to determining that the air quality has not improved after a predetermined number of iterations, a warning may be presented to the user. The warning may indicate that the air treatment component (e.g., light source) is in need of repair. In other instances, the warning may prompt the user to check the fit of their user interface, e.g., to ensure there are no unintentional air leaks in the system.

However, in response to determining that the second AQI value has improved compared to the first AQI value, the method 500 proceeds to operation 510. There, operation 510 includes providing a notification to the user indicating improvements to the AQI value of the treated ambient air compared to the AQI value of the received ambient air. In other words, operation 510 includes informing the user of the improvements to the quality of the air provided to them for inhalation. This will provide useful information to the user, informing them of the benefits of using the air treatment module and respiratory therapy system (or at least the user interface in some configurations. The user may be notified on a display screen of the respiratory therapy device, a message may be pushed to the user's mobile device, etc.

Further steps may be taken to inform the user of additional improvements experienced as a result of using the respiratory therapy system. For instance, the improved quality of air may actually improve a quality of sleep experienced by a user. Accordingly, operation 512 includes correlating the improvements to the AQI value of the treated ambient air with a quality of sleep experienced by the user. As mentioned above, the improved air quality may actually cause the user to experience an improved quality of sleep during subsequent sleep sessions. Thus, by associating the quality of air inspired by a user and the quality of sleep experienced as a result of breathing the higher quality air, a user may be made further aware of the benefits of using the respiratory therapy system. This in turn may give the user further positive reinforcement, increasing the chances of continued use.

The connections between the quality of the air inspired by the user and the quality of sleep experienced by the user may be identified using any desired processes. For instance, machine learning techniques may be used to identify these connections between the quality of the air inspired and the resulting quality of sleep experienced. It follows that in some implementations, a deep learning model may be created (e.g., using supervised and/or unsupervised learning) that is able to identify improvements to the user's quality of sleep based on the specific aspects of the treated air provided to them for inhalation leading up to and/or during a corresponding sleep session.

In response to correlating the improvements to the AQI value of the treated ambient air with a quality of sleep experienced by the user, operation 514 further includes determining a sleep score to assign to the user's sleep session. According to some implementations, the sleep score may be determined by incorporating the AQI value of the treated ambient air and the quality of sleep experienced by the user during a sleep session. For instance, the sleep score may be determined by combining different data readings collected during the sleep session. Different weights may further be applied to the different data readings based on the quality of the air that was provided to the user for inhalation during the sleep session and/or prior to the sleep session. This sleep score may further be compared against previous sleep scores and the user may be notified of improvements resulting from using a respiratory therapy system having the air treatment module.

Additional metrics may also be monitored. For example, the number and/or intensity of medical related treatments conducted by the user throughout the day may be monitored. In another example, medical data of the user (e.g., heart rate, blood pressure, blood sugar levels, etc.) may be monitored. This may allow for the improvements achieved as a result of utilizing a respiratory therapy system having the air treatment module to be further appreciated by the user. Again, this is desirable as the user is provided positive feedback, thereby increasing the likelihood of continued use.

For example, the breathing performance of the user may be monitored during subsequent sleep sessions. By monitoring the breathing performance, the presence of side effect of a medical condition may be identified. For example, the user may be identified as experiencing SDB by monitoring their breathing performance during a subsequent sleep session. In some instances, SDB may be triggered by the presence of certain contaminants in the air breathed by the user. By ensuring the user is provided treated air for inhalation, the SDB may actually be reduced during subsequent sleep sessions.

It follows that medical side effects experienced by users may improve in response to providing treated air for the users to inspire. The severity of a side effect experienced during a given sleep session may thereby be compared against the severity of past side effects experienced by a user, e.g., prior to being provided the treated air for inhalation. As noted above, this desirably allows for the improvements achieved as a result of using the respiratory therapy systems and air treatment modules included herein to be identified and quantified. Moreover, by providing an updated notification to the user indicating improvements to the severity of the side effects they experience may inform the user of enhancements they may not otherwise be aware of. This creates a positive feedback loop which increases the chances of the user continuing to use the respiratory therapy system.

It should also be noted that as used herein, a “sleep session” can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

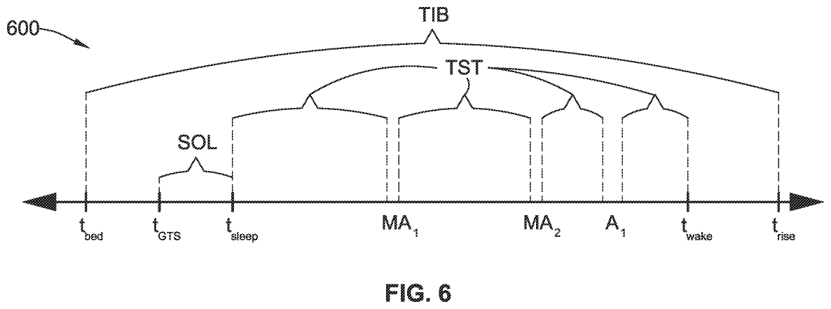

Referring to the timeline 600 in FIG. 6 the enter bed time t_{bed} is associated with the time that the user initially enters the bed (e.g., bed 40 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (tors) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 600 of FIG. 6, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

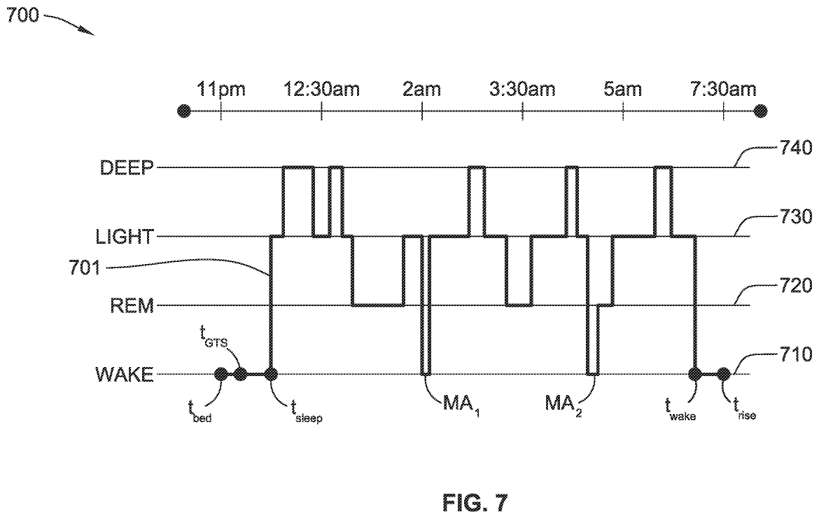

Referring to FIG. 7, an exemplary hypnogram 700 corresponding to the timeline 600 (FIG. 6), according to some implementations, is illustrated. As shown, the hypnogram 700 includes a sleep-wake signal 701, a wakefulness stage axis 710, a REM stage axis 720, a light sleep stage axis 730, and a deep sleep stage axis 740. The intersection between the sleep-wake signal 701 and one of the axes 710-740 is indicative of the sleep stage at any given time during the sleep session.

The sleep-wake signal 701 can be generated based on physiological data associated with the user (e.g., generated by one or more of the sensors 210 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 700 is shown in FIG. 7 as including the light sleep stage axis 730 and the deep sleep stage axis 740, in some implementations, the hypnogram 700 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 204.

The hypnogram 700 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 6), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 6), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (tors), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (tors), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 218, the microphone 220, the camera 232, or any combination thereof. The go-to-sleep time can be determined based on, for example, data from the motion sensor 218 (e.g., data indicative of no movement by the user), data from the camera 232 (e.g., data indicative of no movement by the user and/or that the user has turned off the lights) data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

One or more elements or aspects or steps, or any portion(s) thereof, from one or more of any of claims 1 to 20 below can be combined with one or more elements or aspects or steps, or any portion(s) thereof, from one or more of any of the other claims 1 to 20 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A wearable air treatment system, the system comprising:
a user interface configured to be coupled about at least a portion of a face of a user; and
an air treatment module coupled to the user interface, the air treatment module having:
an air inlet configured to receive ambient air therethrough,
a light source positioned distal of the air inlet and being configured to emit light to treat at least a portion of the received ambient air, and
an air outlet positioned distal of the light source and being configured to direct at least a portion of the treated ambient air towards an interior of the user interface.

2. The wearable air treatment system of claim 1 in combination with a respiratory therapy system, the respiratory therapy system including a conduit and a respiratory therapy device, wherein a first end of the air treatment module is configured to be removably coupled to a first end of the conduit and a second end of the air treatment module is configured to be removably coupled to an inlet of the user interface.

3. The wearable air treatment system of claim 1 in combination with a respiratory therapy system, the respiratory therapy system including a conduit and a respiratory therapy device, wherein a first end of the air treatment module is configured to be removably coupled to a first end of the conduit and a second end of the air treatment module is configured to be removably coupled to an outlet of the respiratory therapy device.

4. The wearable air treatment system of claim 1, the air treatment module further having:
a battery;
a first electrically conductive contact configured to be electrically coupled to a corresponding second electrically conductive contact of the conduit.

5. The wearable air treatment system of claim 4, wherein the battery is a rechargeable battery.

6. The wearable air treatment system of claim 5, wherein the battery is configured to be charged in response to (i) the first electrically conductive contact being electrically coupled to the second electrically conductive contact and (ii) the respiratory therapy device being powered on.

7. The wearable air treatment system of claim 1, wherein the light source is an ultraviolet light source.

8. The wearable air treatment system of claim 1, wherein the at least a portion of the face of the user includes (i) a nose of the user, (ii) a mouth of the user, or (iii) both (i) and (ii).

9. The wearable air treatment system of claim 1, wherein the air treatment module further includes a physical air filter positioned proximal to the air inlet, the physical air filter being configured to remove particulates from the ambient air as it passes through the physical air filter.

10. The wearable air treatment system of claim 9, wherein the physical air filter is one of (i) a high-efficiency particulate absorbing (HEPA) filter or (ii) a scented air filter configured to exude one or more scents.

11. The wearable air treatment system of claim 1, wherein the air treatment module includes a humidifier.

12. The wearable air treatment system of claim 1, wherein a humidifier is removably coupled to and end of the air treatment module.

13. The wearable air treatment system of claim 1, wherein the air treatment module is configured to add one or more scents to the treated ambient air.

14. The wearable air treatment system of claim 1, wherein the air treatment module further includes an ionizer purification component positioned distal of the air inlet and configured to produce positive and negative ions to treat at least a portion of the received ambient air, wherein the ionizer purification component is a plasma air purification component.

15. The wearable air treatment system of claim 1, wherein the air treatment module further includes an activated carbon filter positioned proximal to the air inlet and configured to treat at least a portion of the received ambient air.

16. The wearable air treatment system of claim 1, wherein the air treatment module further includes a polarized-media electronic component positioned distal of the air inlet and configured to generate a polarized electric field to treat at least a portion of the received ambient air.

17. The wearable air treatment system of claim 1, wherein the air treatment module further includes an immobilized cell with a bio-reactive mass positioned distal of the air inlet and configured to treat at least a portion of the received ambient air.

18. The wearable air treatment system of claim 1, the system further comprising:
a memory device having stored thereon machine-readable instructions; and
a control system including one or more processors configured to execute the machine-readable instructions to:
determine a first air quality index (AQI) value corresponding to the received ambient air;
determine a second AQI value corresponding to the treated ambient air;
compare the first AQI value with the second AQI value; and
provide a notification to the user indicating improvements to the AQI value of the treated ambient air compared to the AQI value of the received ambient air.

19. The wearable air treatment system of claim 18, the one or more processors being further configured to execute the machine-readable instructions to:
correlate the improvements to the AQI value of the treated ambient air with a quality of sleep experienced by the user during a subsequent sleep session; and
incorporate the AQI value of the treated ambient air and the quality of sleep experienced by the user in a sleep score assigned to the subsequent sleep session.

20. The wearable air treatment system of claim 18, the one or more processors being further configured to execute the machine-readable instructions to:
monitor breathing performance of the user during a subsequent sleep session;
identify breathing performance that indicates a present side effect of a medical condition;
compare a severity of the present side effect with a severity of past side effects experienced by the user; and
provide an updated notification to the user indicating improvements to the severity of the present side effect caused by the improvements to the AQI value of the treated ambient air.
